(19) United States
(12) Patent Application Publication
(10) Pub. No.: US 2010/0184990 A1
(43) Pub. Date: Jul. 22, 2010

(54) Process for the Manufacture of Flecainide

(76) Inventors: Harmander Pal Singh Chawla, Chandigarh (IN); Anil Shankar Chowdhary, Ahmedabad (IN); Piyush Bhikhubhai Limbad, Ahmedabad (IN); Rajesh Jagannathbhai Jha, Jharkhand (IN); Vipul Narbheshankar Joshi, Porbandar (IN)

Correspondence Address: COOPER & DUNHAM, LLP, 30 Rockefeller Plaza, 20th Floor, NEW YORK, NY, 10112, US

(21) Appl. No.: 11/988,126

(22) PCT Filed: Sep. 9, 2005
(86) PCT No.: PCT/IB2005/002682

§ 371 Date: Mar. 29, 2010

(30) Foreign Application Priority Data
Jul. 4, 2005 (IN) 789/MUM/2005

(51) Int. Cl. C07D 213/46

(52) U.S. Cl. 546/337

(57) ABSTRACT

The invention describes an improved process for the manufacture of a Flecainide intermediate viz N-(pyridin-2-ylmethyl)-2,5-bis(2,2,2-trifluoroethoxy)benzamide (II). It consists of reacting 2,5-bis(2,2,2-trifluoroethoxy)benzoic acid with an acid chloride in a solvent mixture in presence of a base at −10 to −50° C. The resulting mixed anhydride is then condensed with 2-aminomethylpyridine at −10 to −40° C. and the resulting product after aqueous workup is purified by crystallization. This affords the intermediate II in vastly improved yields and quality. The intermediate II on catalytic hydrogenation affords N-(2-piperidinylmethyl)-2,5-bis(2,2,2-trifluoroethoxy)benzamide (Flecainide), isolated as its acetate.

### BACKGROUND OF THE INVENTION

The present invention relates to an improved process for the manufacture of N-(pyridin-2-ylmethyl)2,5-bis(2,2,2-trifluoroethoxy)benzamide II, which is the penultimate intermediate in the manufacture of N-(2-piperidinylmethyl)-2,5-bis(2,2,2-trifluoroethoxy)benzamide acetate (flecainide acetate) of the formula I.

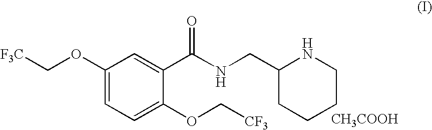

Flecainide acetate (I) is a widely used class 1c antiarrhythmic agent. It is indicated for various types of arrhythmias. It is used to regulate the rate and rhythm of the heart. The heart's pumping action is controlled by electrical signals that pass through the heart muscle. The electrical signals cause the two pairs of heart chambers (left and right arteria and ventricles) to contract in a regular manner to produce regular heartbeats. If the electrical activity in the heart is disturbed for any reason, irregular heartbeats (arrhythmias) of various types can result. These can seriously undermine the pumping action of the heart and result in inefficient blood circulation around the body. Flecainide helps to treat arrhythmias by decreasing the sensitivity of the heart muscle cells to electrical impulses. This regulates the electrical conduction in the heart muscle and reduces disturbances in the heart rhythm. Several processes for the manufacture of the same are reported.

A key intermediate in the synthesis of the compound of the formula I is the compound of the formula II. This intermediate is also the object of the invention of several earlier patents.

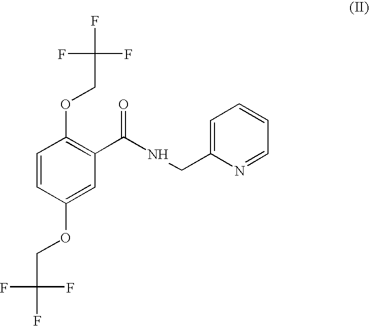

### PRIOR ART

The U.S. Pat. No. 3,900,481 relates to the manufacture of certain compounds in which a carbon atom of a pyrrolidine or piperidine is bonded directly or through a methylene group to the nitrogen of a substituted benzamido group and their pharmaceutically acceptable salts are active as antiarrhythmic agents.

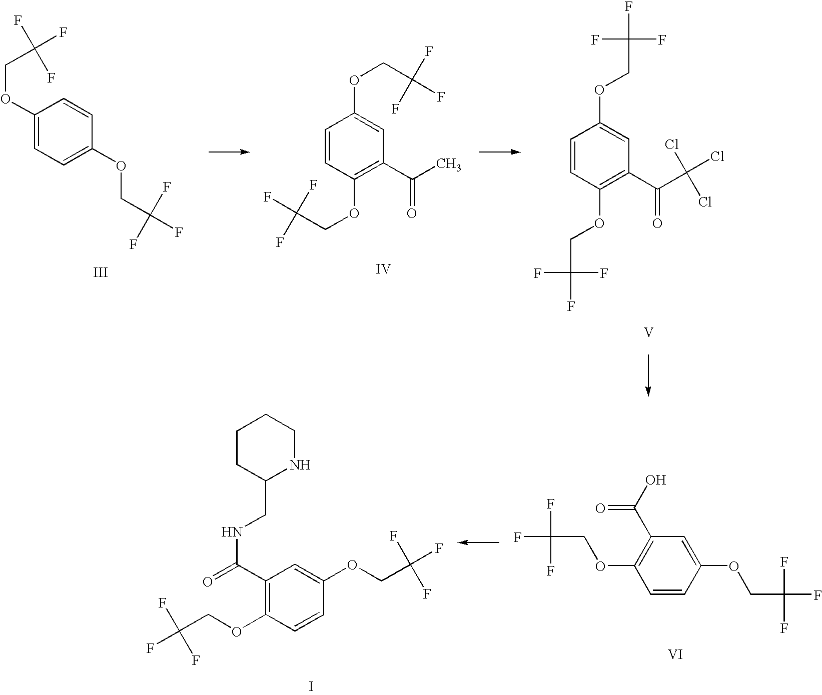

Herein, flecanide was prepared by converting 1,4-R₂C₆H₄ (R═halogen, OH) into 1,4-(F₃CCH₂O)₂C₆H₄ (III), which was acetylated to give 2,5-(F₃CCH₂O)₂C₆H₃COMe (IV), which was then chlorinated to give 2,5-(F₃CCH₂O)₂C₆H₃COCCl₃ (V), which was finally hydrolyzed to (F₃CCH₂O)₂C₆H₃CO₂H (VI) and was converted to acid chloride followed by reaction with 2-aminomethylpiperidine give I. This process utilizes hazardous chemicals and intermediates, which are commercially not available from multiple sources and is hence not of commercial importance. Besides this process gives the compound in low purifies due to coupling occurring at the piperidinyl nitrogen and is hence not economical and commercially viable.

The U.S. Pat. No. 4,097,481 and U.S. Pat. No. 4,617,396 deal with tertiary amide derivatives of pyrrolidine and piperidine and process for their preparation but essentially having a similar strategy for amidification as described for U.S. Pat. No. 3,900,481. These processes suffer similarly as detailed above.

The U.S. Pat. No. 4,642,384 relates to processes for the preparation of intermediates such as 2,5-bis(2,2,2-trifluoroethoxy)acetophenone, 2,5-bis(2,2,2-trifluoroethoxy)α,α-dichloroacetophenone, 2,5-bis(2,2,2-trifluoroethoxy)α,α,α-trichloroacetophenone and subsequent condensation with 2-aminomethylpyridine followed by catalytic hydrogenation to afford Flecainide acetate of the formula I. This process is again not advantageous on an industrial scale.

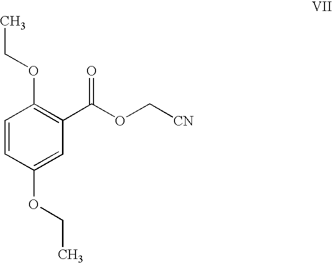

The patent U.S. Pat. No. 6,316,627 provides with a process for preparation of the title compound characterized in that the product namely, 2,5-bis(2,2,2-trifluoroethoxy)benzoic acid was reacted with XCH₂CN to form a cyanomethyl ester derivative (VII) and was converted to flecainide by reacting with RCH₂NH₂ (where R denotes pyridyl group) followed by catalytic hydrogenation. This patent makes use of expensive haloalkyl nitriles for forming the intermediate and is thus far from satisfactory.

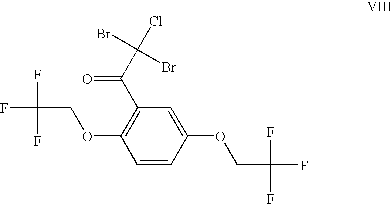

The patent U.S. Pat. No. 6,458,957 related to the use of α,α-dibromo-α-chloroacetophenone compounds (VIII), more particularly 2,5-bis(2,2,2-trifluoroethoxy)-α,α-dibromo-α-chloroacetophenone as intermediates for preparing Flecainide. The process comprises of converting the α-chloro acetophenone to an α,α-dibromo-α-chloroacetophenone derivative and reacting the same with a primary or secondary amine. The process is further characterized in that when the amine is 2-(aminomethyl)pyridine it forms 2,5-bis(2,2,2trifluoroethoxy)-N-(2-pyridylmethyl)benzamide and reducing the same with hydrogen affords 2,5-bis(2,2,2 trifluoroethoxy)-N-(2-piperidylmethyl)benzamide. This process involves bromination and is hence unsatisfactory from an ecological point of view.

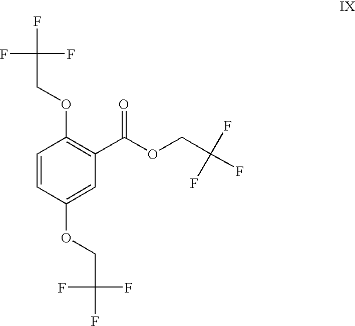

The patent U.S. Pat. No. 6,599,992 relates to a process for the preparation of flecainide comprising of synthesis of the key intermediate 2′,2′,2′-trifluoroethanol 2,5-bis-(2,2,2-trifluooroethoxy)benzoate (IX), by reaction of 2,5-dihydroxy benzoic acid with 2,2,2-trifluoroethanol perfluorobutanesulphonate in presence of organic bases. This intermediate on subsequent reaction with 2-aminomethylpiperidine gave flecainide. This process again utilizes the costly trifluoroethanol as a leaving group and is hence economically unviable.

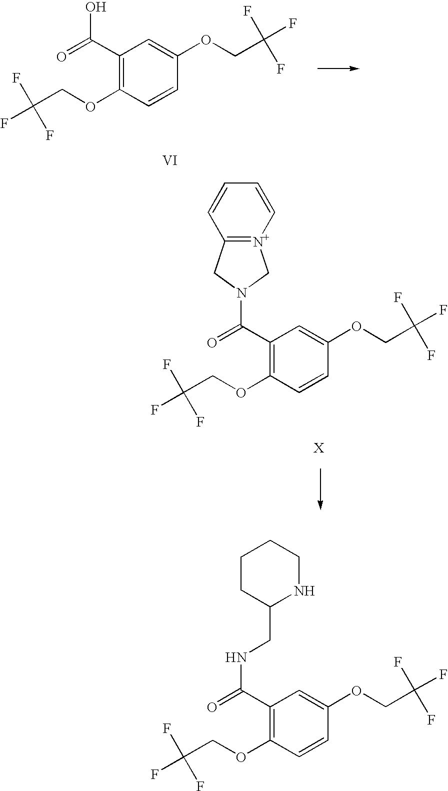

The Spanish patent ES 2007,02 relates to a process for the manufacture of Flecainide comprising of reacting an activated derivative (X) of 2,5-bis(2,2,2-trifluoroethoxy)benzoic acid (X) with 2-azaindolizidine, which is selectively hydrolyzed to flecainide followed by salification with glacial acetic acid. This route again employs a costly chemical for activation and hence not feasible on a commercial scale.

### SUMMARY OF THE INVENTION

An object of the invention is to provide a process for the transformation from a compound of the formula X to a compound of the formula II and thereby to the compound of the formula I in high yields and purities.

Another object of the present invention is to provide a process for the manufacture of the compound of the formula II that is simple, easy and convenient to carry out.

Another object of the invention is to provide a process for the manufacture of the compound of the formula H that is economical and commercially viable. The intermediate can then be subjected to catalytic hydrogenation for the reduction of the pyridyl group to afford flecainide, which is isolated as the acetate.

### DETAILED DESCRIPTION

According to the invention there is provided a process for the manufacture of N-(pyridin-2-ylmethyl)-2,5-bis(2,2,2-trifluoroethoxy)benzamide (II) consisting of reacting 2,5-bis(2,2,2-trifluoroethoxy)benzoic acid of the formula VI with an acid chloride of the formula R₁COCl wherein R1 is ethyl, tertiary butyl, or ethoxy in a suitable solvent mixture in the presence of a base at −10 to −50° C. to obtain a mixed anhydride of the formula (XI)

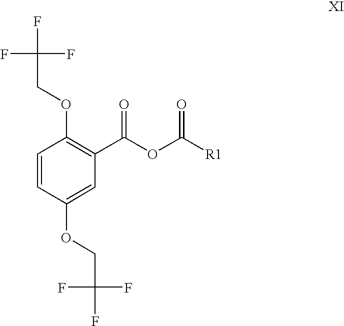

wherein R1 is as defined above which is then condensed with a solution of 2-aminomethyl pyridine at −10 to −40° C. followed by hydrolytic workup and extraction of the product and isolation by distillation of the extracting solvent.

As an acid chloride one can utilize methyl chloroformate, ethyl chloroformate, or trimethyl acetyl chloride (pivaloyl chloride) preferably pivaloyl chloride.

As a base one can utilize the tertiary amines such as triethyl amine, pyridine, lutidine or N-methyl morpholine preferably triethyl amine.

The suitable solvent mixture comprises a halogenated hydrocarbon solvent such as methylene chloride, chloroform or ethylene chloride preferably methylene chloride and a polar aprotic solvent selected from the group comprising N,N-dimethyl formamide, N,N-dimethyl acetamide, N,N-dimethyl sulfoxide or N-methyl-2-pyrrolidone preferably N,N-dimethyl acetamide. The halogenated hydrocarbon solvent and polar aprotic solvent are preferably in the ratio 3:1.

The base used for the mixed anhydride formation is employed in the molar ratio from 1:0.9 to 1:1.5 with respect to the compound of the formula VI preferably 1:1.05.

The temperature during the mixed anhydride stage is in the range from −10 to −50° C. preferably −20° C.

Typically the condensation reaction of the mixed anhydride with 2-aminomethyl pyridine is conducted from −10 to −40° C., preferably about −20° C.

The process of the invention does not use any corrosive chemical. The condensation reaction between the mixed anhydride and 2-aminomethyl pyridine is practically quantitative and affords the compound of the formula II in high yields (˜95%) and high purities (˜99%).

Therefore the process of the invention is simple, easy, convenient and fast to carry out. For these reasons the process is economical and commercially viable.

The following examples are illustrative of the invention but not restricted to the scope thereof.

### EXAMPLE-1

60 gm of 2,5-bis (2,2,2-trifluoroethoxy)benzoic acid was suspended in a mixture of 480 ml methylene chloride and 90 ml N,N-dimethyl acetamide. To this 22.9 gm of triethyl amine was added and the mixture was cooled to −30° C. To this 27.4 gm of pivaloyl chloride and the mass stirred at −20 to −30° C. A solution of 21.6 gm of 2-aminomethyl pyridine in 120 ml of methylene chloride was added and the mixture was maintained at −20° C. for 120 min. The reaction mixture was quenched in 780 ml of water. After 30 min the lower organic layer was separated and washed with 300 ml 5% sodium carbonate solution. The organic layer was filtered and distilled. The residue was diluted with 360 ml cyclohexane and filtered. The solid was washed with 100 ml cyclohexane and dried at 40° C. under vacuum to give 72 gm of N-(pyridin-2-ylmethyl)-2,5-bis(2,2,2-trifluoroethoxy)benzamide.

Water (by Karl Fisher)=0.23%. HPLC assay=99.2%. (Theoretical=75 gm)

### EXAMPLE-2

The procedure of example 1 was followed with 26 gm of N-methyl morpholine instead of triethyl amine and the isolated product dried at 40° C. under vacuum to give 71 gm of N-(pyridin-2-ylmethyl)-2,5-bis(2,2,2-trifluoroethoxy)benzamide.

Water (by Karl Fisher)=0.18%. HPLC assay=99.1%. (Theoretical=75 gm)

### EXAMPLE-3

The procedure of example 1 was followed with 24.7 gm of ethyl chloro formate instead of pivaloyl chloride and the isolated product dried at 40° C. under vacuum to give 71.5 gm of N-(pyridin-2-ylmethyl)-2,5-bis(2,2,2-trifluoroethoxy)benzamide.

Water (by Karl Fisher)=0.26%. HPLC assay=99.0%. (Theoretical=75 gm)

### EXAMPLE-4

The procedure of example 1 was followed with 90 ml of N,N-dimethyl formamide instead of N,N-dimethyl acetamide and the isolated product dried at 40° C. under vacuum to give 70.8 gm of N-(pyridin-2-ylmethyl)-2,5-bis(2,2,2-trifluoroethoxy)benzamide.

Water (by Karl Fisher)=0.29%. HPLC assay=99.1%. (Theoretical=75 gm)

1. A process for the manufacture of N-(pyridin-2-ylmethyl)-2,5-bis(2,2,2-trifluoroethoxy)benzamide of the formula II, 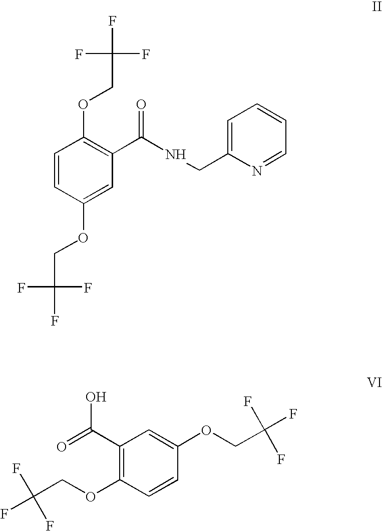
comprising of reacting 2,5-bis(2,2,2-trifluoroethoxy)benzoic acid of the formula VI with an acid chloride of the formula R₁COCl wherein R₁ is methyl, ethyl, t-butyl or ethoxy in a solvent mixture, in the presence of a base at −10 to −50° C. to obtain a mixed anhydride of the formula XI 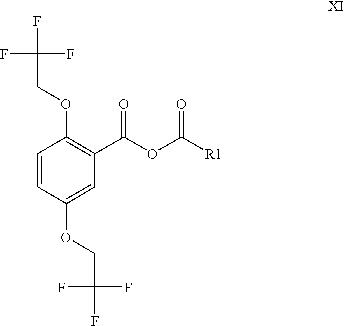
where R₁ is methyl, ethyl, t-butyl or ethoxy, the mixed anhydride is then condensed with a solution of 2-aminomethyl pyridine at −10 to −40° C. and subsequently subjected to hydrolytic workup and extraction followed by distillation of the solvent to afford the desired compound of the formula II.

2. A process as claimed in claim 1, wherein the acid chloride can be selected from among the group comprising of methyl chloroformate, ethyl chloroformate, or trimethyl acetyl chloride (pivaloyl chloride) but preferably pivaloyl chloride and the solvent mixture comprises methylene chloride and N,N-dimethyl acetamide.

3. A process as claimed in claim 1, wherein the solvent mixture comprises of a halogenated hydrocarbon solvent such as methylene chloride, chloroform or ethylene chloride preferably methylene chloride and a polar aprotic solvent selected from the group comprising N,N-dimethyl formamide, N,N-dimethyl acetamide, N,N-dimethyl sulfoxide or N-methyl-2-pyrrolidone preferably N,N-dimethyl acetamide.

4. A process as claimed in claim 1, wherein the base can be selected from the group comprising of tertiary amines such as triethyl amine, pyridine, lutidine or N-methyl morpholine preferably triethyl amine.

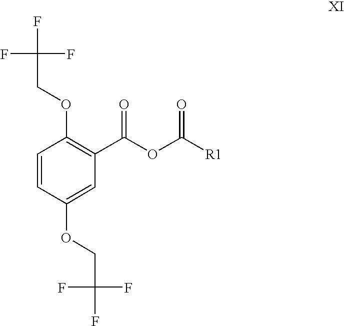

5. A process as claimed in claim 1, wherein the temperature during the mixed anhydride stage is in the range from −10 to -50° C. preferably −20° C.

6. A process as claimed in claim 1, wherein the temperature during the condensation stage is in the range from −10 to −40° C. preferably −20° C.

7. A process as claimed in claim 4, wherein the solvent ratio of the halogenated hydrocarbon to the polar aprotic solvent ranges from 1:5 to 1:10 preferably 1:6.

8. A process as claimed in claim 4, wherein the base used for the mixed anhydride formation is employed in the molar ratio from 1:0.9 to 1:1.5 with respect to the compound of the formula VI preferably 1:1.05.

9. A process as claimed in claim 4, wherein the compound of the formula II is converted to a compound of the formula I by catalytic hydrogenation.

10. A process for the manufacture of N-(pyridin-2-ylmethyl)-2,5-bis(2,2,2-trifluoroethoxy)benzamide of the formula H and the base used for the mixed anhydride formation substantially as herein described with reference to the given specification.
